(19) United States
(12) Patent Application Publication
(10) Pub. No.: US 2016/0228485 A1
(43) Pub. Date: Aug. 11, 2016

(54) SKIN MEDICATING SYSTEM

(71) Applicant: Latasha M. Watts, Cincinnati, OH (US)

(72) Inventor: Latasha M. Watts, Cincinnati, OH (US)

(21) Appl. No.: 14/620,165

(22) Filed: Feb. 11, 2015

(51) Int. Cl. A61K 36/886; A61K 31/573; A61K 35/02; A61K 36/185

(52) U.S. Cl. CPC A61K 36/886; A61K 36/185; A61K 31/573; A61K 35/02

(57) ABSTRACT

A skin medicating system for providing a plurality of components to be mixed to treat various skin ailments. The system includes a plurality of basic components. A first basic component comprises one part a mixture of eighty to ninety percent witch hazel extract and ten to twenty percent alcohol, one part aloe-vera extract, and one part petrolatum by volume. A second basic component comprises a cocoa butter crème lotion. A third basic component comprises a betamethansonate valerate crème .

### BACKGROUND OF THE DISCLOSURE

### Field of the Disclosure

The disclosure relates to skin medicating devices and more particularly pertains to a new skin medicating device for providing a plurality of components to be mixed to treat various skin ailments.

### SUMMARY OF THE DISCLOSURE

An embodiment of the disclosure meets the needs presented above by generally comprising a plurality of basic components. A first basic component comprises petrolatum. A second basic component comprises a cocoa butter crème lotion. A third basic component comprises a betamethansonate valerate crème .

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### DESCRIPTION OF THE PREFERRED EMBODIMENT

A new skin medicating system embodying the principles and concepts of an embodiment of the disclosure will be described. The skin medicating system generally comprises a plurality of basic composition components which are mixed together in various combinations depending on the type of ailment to be treated.

A first basic component comprises petrolatum. A second basic component comprises a cocoa butter crème lotion of the type commercially available. The ingredients of the cocoa butter crème lotion include deionized water, mineral oil, stearic acid, cetyl glycol, phenoxyethanol, isobutylparaben, n-butylparaben, carbomer, disodium EDTH, Tocopheryl acetate, retinyl pamitate, and cocoa butter. A third basic component is betamethansonate valerate foam comprising between 1.0 and 1.4 milligrams betamethasone valerate, USP, per gram of a thermolabile hydroethanolic foam vehicle comprising cetyl, citric acid, between 58% and 62% ethanol, polysorbate 60, potassium citrate, propylene glycol, purified water, and stearyl alcohol pressurized with a hydrocarbon propellant such as propane or butane. The third basic component may be found commercially under the trade name LUXIQ.

The three basic components are provided in separate containers and may be mixed as needed to treat various maladies. A first mixture comprises equal parts of each of the first basic component, the second basic component, and the third basic component. The first mixture is used for treatment of soreness and pain associated with lupus, eczema, cuts, abrasions, and burns. When treating skin the first mixture may further include the addition of olive oil. A second mixture comprises equal parts of the first component, the second component, the third component, and 100% tea tree oil. The second mixture is used to facilitate hair growth and treat psoriasis. A third mixture is formed by using one half part of the first mixture to one part 100% aloe vera by volume. The third mixture is used for infants having maladies treated by use of the first mixture.

In use, the three basic components are mixed as described above and applied to affected areas of the skin as needed. Hands should be washed after each application.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A skin medicating system comprising:
a plurality of basic components, said basic components comprising a first basic component, a second basic component, and a third basic component;
said first basic component comprising petrolatum;
said second basic component comprising a cocoa butter crème lotion; and
said third basic component comprising a betamethansonate valerate foam.

2. The system of claim 1, further comprising said first basic component, said second basic component, and said third basic component being mixed in equal parts to define a first mixture.

3. The system of claim 2, further comprising said first mixture being combined with tea tree oil in an amount equal to each part of said first component, said second component and said third component.

4. The system of claim 2, further comprising one part of said first mixture being combined with one part of aloe vera by volume.

5. The system of claim 1, further comprising said second basic component comprising deionized water, mineral oil, stearic acid, cetyl glycol, phenoxyethanol, isobutylparaben, n-butylparaben, carbomer, disodium EDTH, Tocopheryl acetate, retinyl pamitate, and cocoa butter.

6. The system of claim 5, further comprising said third basic component comprising between 1.0 and 1.4 milligrams betamethasone valerate, USP, per gram of a thermolabile hydroethanolic foam vehicle comprising cetyl, citric acid, between 58% and 62% ethanol, polysorbate 60, potassium citrate, propylene glycol, purified water, and stearyl alcohol pressurized with a hydrocarbon propellant.

7. A skin medicating system comprising:
a plurality of basic components, said basic components comprising a first basic component, a second basic component, and a third basic component;
said first basic component comprising petrolatum;
said second basic component comprising a cocoa butter crème lotion comprising deionized water, mineral oil, stearic acid, cetyl glycol, phenoxyethanol, isobutylparaben, n-butylparaben, carbomer, disodium EDTH, Tocopheryl acetate, retinyl pamitate, and cocoa butter; and
said third basic component comprising a betamethansonate valerate crème comprising between 1.0 and 1.4 milligrams betamethasone valerate, USP, per gram of a thermolabile hydroethanolic foam vehicle comprising cetyl, citric acid, between 58% and 62% ethanol, polysorbate 60, potassium citrate, propylene glycol, purified water, and stearyl alcohol pressurized with a hydrocarbon propellant.
